(19) United States
(12) Patent Application Publication
(10) Pub. No.: US 2021/0206773 A1
(43) Pub. Date: Jul. 8, 2021

(54) DRUG FOR TREATING DISORDERS OF CORNEAL EPITHELIUM

(71) Applicant: EA PHARMA CO., LTD., Tokyo (JP)

(72) Inventors: Tohru MIZUSHIMA, Minato-ku (JP); Hiroshi IWATA, Chuo-ku (JP)

(73) Assignee: EA PHARMA CO., LTD., Tokyo (JP)

(21) Appl. No.: 17/212,707

(22) Filed: Mar. 25, 2021

Related U.S. Application Data
(63) Continuation of application No. 15/539,015, filed on Jun. 22, 2017, now abandoned.

(30) Foreign Application Priority Data
Dec. 22, 2014 (JP) 2014-258383

(51) Int. Cl. C07D 487/04; A61K 31/5517; A61P 27/02; A61K 31/551

(52) U.S. Cl. CPC C07D 487/04; A61K 31/551; A61P 27/02; A61K 31/5517

(57) ABSTRACT

A new agent for the treatment of a corneal epithelium disorder, which acts directly on corneal epithelial cells, is provided. Disclosed is an agent for the treatment of a corneal epithelium disorder, the agent comprising a compound of Formula (1) or a salt thereof as an active ingredient:

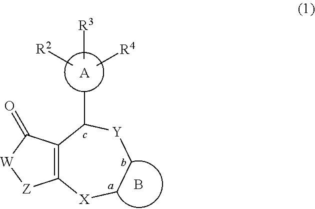

wherein A is an aromatic ring, a heterocyclic ring, or an aliphatic ring; R², R³, and R⁴, which may be identical or different, each independently are, for example, a hydrogen atom, a halogen atom; B is an aromatic ring which may have a substituent, a heterocyclic ring which may have a substituent, or an aliphatic ring which may have a substituent; —X—, —Y—, and —Z—, which may be identical or different, each independently are O—, —NH—, —NR⁵—, —S—, —SO—, —SO₂—, —CH₂—, —CR⁶R⁷—, or —CO—; and —W— is —NR¹—, —O—, or —CR⁸R⁹—.

### FIELD OF THE INVENTION

The present invention relates to an agent for the treatment of corneal epithelium disorders.

### BACKGROUND OF THE INVENTION

Corneal epithelium disorders are diseases associated with eye discomfort or visual dysfunction caused by various factors, such as dry eye, inappropriate use of contact lenses, antiseptic agents included in eye drops, and autoimmune diseases including Sjögren's syndrome. Among these, particularly the number of dry eye patients is said to be about 22 millions in Japan, and the number of patients who suffer from corneal epithelium disorders tends to increase. Further, the prevalence rate increases with aging.

In regard to dry eye, it is considered that the lacrimal fluid becomes hyperosmotic as a result of reduced secretion and increased evaporation of the lacrimal fluid, inflammatory response and cell injury occur in the cornea, and the lacrimal fluid layer is unstabilized, so that as a result, the onset of corneal epithelium disorders proceeds (Non-Patent Literature 1).

Regarding the method for the treatment of dry eye currently used, for example, lacrimal fluid replacement therapy using sodium hyaluronate, diquafosol sodium (acceleration of mucin secretion), and rebamipide (acceleration of mucin production); use of spectacles or goggles; and lacrimal opening plug method are used.

### CITATION LIST

### Patent Literature

Patent Literature 1: WO 2002/044180 A

### Non Patent Literature

Non-Patent Literature 1: Website homepage of Japanese Ophthalmological Society

### SUMMARY OF INVENTION

### Technical Problem

However, in regard to the conventional method for treatment of dry eye, the main purpose lies in replacement of lacrimal fluid or prevention of drying, and the method for the treatment does not act directly on the corneal epithelial cells. Thus, it is hard to say that a sufficient therapeutic effect is obtained therefrom. Furthermore, under the circumstances, sufficient treatment is not achieved for corneal epithelium disorders that occur as a result of diabetes mellitus or long-term use of eye drops.

Therefore, an object of the present invention is to provide a new agent for the treatment of corneal epithelium disorders, which acts directly on corneal epithelial cells.

### Solution to Problem

Thus, the inventors of the present invention conducted extensive investigations in search of compounds having protective action on corneal epithelial cells with inflammation or cell injury, in connection with dry eye, for example. As a result, totally unexpectedly, the inventors found that some lactam compounds that are known to have sugar transport enhancement action and hypoglycemic action, strongly suppress a decrease in the survival rate of corneal epithelial cells caused by hyperosmotic stress, and this action is based on the action of corneal epithelial cells for suppressing apoptosis under hyperosmotic stress; and that since the lactam compounds prevent corneal epithelial cell injury and also increase the amount of lacrimal fluid or mucin, the lactam compounds are useful as agents for the treatment of corneal epithelium disorders caused by dry eye, for example. Thus, the inventors completed the present invention.

That is, the present invention provides the following items [1] to [28].

[1] An agent for the treatment of a corneal epithelium disorder, the agent comprising a compound of Formula (1) or a salt thereof as an active ingredient:

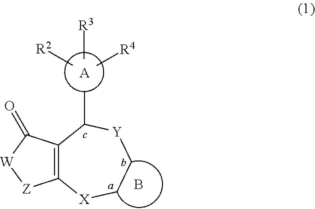

wherein A is an aromatic ring, a heterocyclic ring, or an aliphatic ring; R², R³, and R⁴, which may be identical or different, each independently are a hydrogen atom, a halogen atom, a hydroxy group, an alkyl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an alkenyl group which may have a substituent, an alkynyl group which may have a substituent, an aryl group which may have a substituent, a heteroaryl group which may have a substituent, a benzyloxy group which may have a substituent, an aryloxy group which may have a substituent, a heteroaryloxy group which may have a substituent, an arylamino group which may have a substituent, an arylvinyl group which may have a substituent, or an arylethynyl group which may have a substituent; B is an aromatic ring which may have a substituent, a heterocyclic ring which may have a substituent, or an aliphatic ring which may have a substituent; —X—, —Y—, and —Z—, which may be identical or different, each independently are —O—, —NH—, —NR⁵—, —S—, —SO—, —SO₂—, —CH₂—, —CR⁶R⁷—, or —CO— (wherein R⁵ is a lower alkyl group which may have a substituent, an acyl group which may have a substituent, an alkoxycarbonyl group which may have a substituent, a carbamoyl group which may have a substituent, or a sulfonyl group which may have a substituent; and R⁶ and R⁷, which may be identical or different, each independently are a hydrogen atom, a halogen atom, a hydroxy group, an alkyl group which may have a substituent, an aryl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, or a trifluoromethyl group); —W— is —NR¹—, —O—, or —CR⁸R⁹— (wherein R¹ is a hydrogen atom, a lower alkyl group which may have a substituent, or an aryl group which may have a substituent; and R⁸ and R⁹, which may be identical or different, each independently are a hydrogen atom, a halogen atom, a hydroxy group, an alkyl group, an aryl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, or a trifluoromethyl group); and a, b, and c each represent the position of a carbon atom; provided that:

(i) the substituent is selected from the group consisting of a halogen atom, a hydroxy group, an alkyl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an aryl group, and a heteroaryl group;

(ii) when B is a benzene ring, —X— and —Y— both represent —NH—, —Z— is —CH₂—, and —W— is —NH—, —A(R²)(R³)(R⁴) is none of a phenyl group, a 4-bromophenyl group, a 4-hydroxyphenyl group, a 4-methoxyphenyl group, a 2-hydroxyphenyl group, a 3,4-dimethoxyphenyl group, or a 3-methoxy-4-hydroxyphenyl group;

(iii) when B is a benzene ring, —X— is —NH—, —CR⁶R⁷—, and —W— is —NH—, neither of R⁶ and R⁷ is a methyl group;

(iv) when B is a benzene ring, —X— is —NH—, —Z— is —CO—, and —W— is —NR¹—, R¹ is not a p-tolyl group;

(v) when B is a benzene ring, —X— is —NH—, —Y— is —S—, —Z— is —CR⁶R⁷—, and —W— is —NH—, neither of R⁶ and R⁷ is a methyl group; and

(vi) when B is a benzene ring, —X— is —NH—, —Y— is —S—, and —Z— is —CH₂—, —W— is not —O—.

[2] The agent for the treatment of a corneal epithelium disorder according to [1], wherein in Formula (1), A is a benzene ring; B is a cyclohexane ring; W is —NR¹—; X is —NH—; Y is —NR⁵—; and Z is —CH₂—.

[3] The agent for the treatment of a corneal epithelium disorder according to [2], wherein R¹ is H, and R⁵ is an acyl group which may have a substituent.

[4] The agent for the treatment of a corneal epithelium disorder according to any one of [1] to [3], wherein the compound of Formula (1) is a compound of the following Formula (1a):

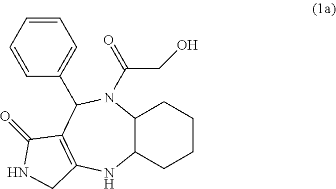 [SMILES: O=C1NCC2=C1C(c1ccccc1)N(C(=O)CO)C1CCCCC1N2]

[5] The agent for a corneal epithelium disorder according to any one of [1] to [4], wherein the agent is an eye drop.

[6] The agent for the treatment of a corneal epithelium disorder according to any one of [1] to [5], wherein the corneal epithelium disorder is dry eye.

[7] A corneal epithelial cell apoptosis inhibitor comprising a compound of Formula (1) or a salt thereof as an active ingredient:

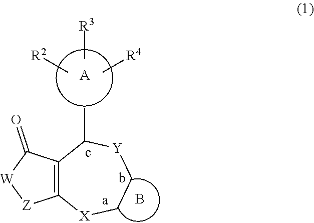

wherein A is an aromatic ring, a heterocyclic ring, or an aliphatic ring; R², R³, and R⁴, which may be identical or different, each independently are a hydrogen atom, a halogen atom, a hydroxy group, an alkyl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an alkenyl group which may have a substituent, an alkynyl group which may have a substituent, an aryl group which may have a substituent, a heteroaryl group which may have a substituent, a benzyloxy group which may have a substituent, an aryloxy group which may have a substituent, a heteroaryloxy group which may have a substituent, an arylamino group which may have a substituent, an arylvinyl group which may have a substituent, or an arylethynyl group which may have a substituent; B is an aromatic ring which may have a substituent, a heterocyclic ring which may have a substituent, or an aliphatic ring which may have a substituent; —X—, —Y—, and —Z—, which may be identical or different, each independently are —O—, —NH—, —NR⁵—, —S—, —SO—, —SO₂—, —CH₂—, —CR⁶R⁷—, or —CO— (wherein R⁵ is a lower alkyl group which may have a substituent, an acyl group which may have a substituent, an alkoxycarbonyl group which may have a substituent, a carbamoyl group which may have a substituent, or a sulfonyl group which may have a substituent; and R⁶ and R⁷, which may be identical or different, each independently are a hydrogen atom, a halogen atom, a hydroxy group, an alkyl group which may have a substituent, an aryl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, or a trifluoromethyl group); —W— is —NR¹—, —O—, or —CR⁸R⁹— (wherein R¹ is a hydrogen atom, a lower alkyl group which may have a substituent, or an aryl group which may have a substituent; and R⁸ and R⁹, which may be identical or different, each independently are a hydrogen atom, a halogen atom, a hydroxy group, an alkyl group, an aryl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, or a trifluoromethyl group); and a, b, and c each represent the position of a carbon atom; provided that:

(i) the substituent is selected from the group consisting of a halogen atom, a hydroxy group, an alkyl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an aryl group, and a heteroaryl group;

(ii) when B is a benzene ring, —X— and —Y— both represent —NH—, —Z— is —CH₂—, and —W— is —NH—, —A(R²)(R³)(R⁴) is none of a phenyl group, a 4-bromophenyl group, a 4-hydroxyphenyl group, a 4-methoxyphenyl group, a 2-hydroxyphenyl group, a 3,4-dimethoxyphenyl group, or a 3-methoxy-4-hydroxyphenyl group;

(iii) when B is a benzene ring; —X— is —NH—, —Z— is —CR⁶R⁷— and —W— is —NH—, neither of R⁶ and R⁷ is a methyl group;

(iv) when B is a benzene ring, —X— is —NH—, —Z— is —CO—, and —W— is —NR¹—, R¹ is not a p-tolyl group;

(v) when B is a benzene ring, —X— is —NH—, —Y— is —S—, —Z— is —CR⁶R⁷— and —W— is —NH—, neither of R⁶ and R⁷ is a methyl group; and

(vi) when B is a benzene ring, —X— is —NH—, —Y— is —S—, and —Z— is —CH₂—, —W— is not —O—.

[8] Use of a compound of Formula (1) or a salt thereof for the production of an agent for the treatment of a corneal epithelium disorder:

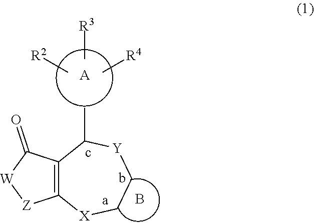

wherein A is an aromatic ring, a heterocyclic ring, or an aliphatic ring; R², R³, and R⁴, which may be identical or different from each other, each independently is a hydrogen atom, a halogen atom, a hydroxy group, an alkyl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an alkenyl group which may have a substituent, an alkynyl group which may have a substituent, an aryl group which may have a substituent, a heteroaryl group which may have a substituent, a benzyloxy group which may have a substituent, an aryloxy group which may have a substituent, a heteroaryloxy group which may have a substituent, an arylamino group which may have a substituent, an arylvinyl group which may have a substituent, or an arylethynyl group which may have a substituent; B is an aromatic ring which may have a substituent, a heterocyclic ring which may have a substituent, or an aliphatic ring which may have a substituent; —X—, —Y—, and —Z—, which may be identical or different, each independently are —O—, —NH—, —NR⁵—, —S—, —SO—, —SO₂—, —CH₂—, —CR⁶R⁷—, or —CO— (wherein R⁵ is a lower alkyl group which may have a substituent, an acyl group which may have a substituent, an alkoxycarbonyl group which may have a substituent, a carbamoyl group which may have a substituent, or a sulfonyl group which may have a substituent; and R⁶ and R⁷, which may be identical or different, each independently are a hydrogen atom, a halogen atom, a hydroxy group, an alkyl group which may have a substituent, an aryl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, or a trifluoromethyl group); —W— is —NR¹—, —O—, or —CR⁸R⁹— (wherein R¹ is a hydrogen atom, a lower alkyl group which may have a substituent, or an aryl group which may have a substituent; and R⁸ and R⁹, which may be identical or different, each independently are a hydrogen atom, a halogen atom, a hydroxy group, an alkyl group, an aryl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, or a trifluoromethyl group); and a, b, and c each represent the position of a carbon atom, provided that:

(i) the substituent is selected from the group consisting of a halogen atom, a hydroxy group, an alkyl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an aryl group, and a heteroaryl group;

(ii) when B is a benzene ring, —X— and —Y— both represent —NH—, —Z— is —CH₂—, and —W— is —NH—, —A(R²)(R³)(R⁴) is none of a phenyl group, a 4-bromophenyl group, a 4-hydroxyphenyl group, a 4-methoxyphenyl group, a 2-hydroxyphenyl group, a 3,4-dimethoxyphenyl group, or a 3-methoxy-4-hydroxyphenyl group;

(iii) when B is a benzene ring, —X— is —NH—, —Z— is —CR⁶R⁷—, and —W— is —NH—, neither of R⁶ and R⁷ is a methyl group;

(iv) when B is a benzene ring, —X— is —NH—, —Z— is —CO—, and —W— is —NR¹—, R¹ is not a p-tolyl group;

(v) when B is a benzene ring, —X— is —NH—, —Y— is —S—, —Z— is —CR⁶R⁷—, and —W— is —NH—, neither of R⁶ and R⁷ is a methyl group; and

(vi) when B is a benzene ring, —X— is —NH—, —Y— is —S—, and —Z— is —CH₂—, —W— is not —O—.

[9] The use according to [8], wherein in Formula (1), A is a benzene ring; B is a cyclohexane ring; W is —NR¹—; X is —NH—; Y is —NR⁵—; and Z is —CH₂—.

[10] The use according to [9], wherein R¹ is H, and R⁵ is an acyl group which may have a substituent.

[11] The use according to any one of [8] to [10], wherein the compound of Formula (1) is a compound of the following Formula (1a):

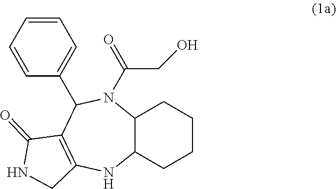 [SMILES: O=C1NCC2=C1C(c1ccccc1)N(C(=O)CO)C1CCCCC1N2]

[12] The use according to any one of [8] to [11], wherein the use is an eye drop.

[13] The use according to any one of [8] to [12], wherein the corneal epithelium disorder is dry eye.

[14] Use of a compound of Formula (1) or a salt thereof for the production of a corneal epithelial cell apoptosis inhibitor:

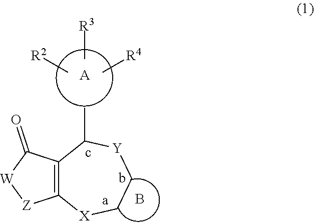

wherein A is an aromatic ring, a heterocyclic ring, or an aliphatic ring; R², R³, and R⁴, which may be identical or different, each independently are a hydrogen atom, a halogen atom, a hydroxy group, an alkyl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an alkenyl group which may have a substituent, an alkynyl group which may have a substituent, an aryl group which may have a substituent, a heteroaryl group which may have a substituent, a benzyloxy group which may have a substituent, an aryloxy group which may have a substituent, a heteroaryloxy group which may have a substituent, an arylamino group which may have a substituent, an arylvinyl group which may have a substituent, or an arylethynyl group which may have a substituent; B is an aromatic ring which may have a substituent, a heterocyclic ring which may have a substituent, or an aliphatic ring which may have a substituent; —X—, —Y—, and —Z—, which may be identical or different, each independently are —O—, —NH—, —NR⁵—, —S—, —SO—, —SO₂—, —CH₂—, —CR⁶R⁷—, or —CO— (wherein R⁵ is a lower alkyl group which may have a substituent, an acyl group which may have a substituent, an alkoxycarbonyl group which may have a substituent, a carbamoyl group which may have a substituent, or a sulfonyl group which may have a substituent; and R⁶ and R⁷, which may be identical or different, each independently are a hydrogen atom, a halogen atom, a hydroxy group, an alkyl group which may have a substituent, an aryl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, or a trifluoromethyl group); —NR¹—, —O—, or —CR⁸R⁹— (wherein R¹ is a hydrogen atom, a lower alkyl group which may have a substituent, or an aryl group which may have a substituent; and R⁸ and R⁹, which maybe identical or different, each independently are a hydrogen atom, a halogen atom, a hydroxy group, an alkyl group, an aryl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, or a trifluoromethyl group); and a, b, and c each represent the position of a carbon atom; provided that:

(i) the substituent is selected from the group consisting of a halogen atom, a hydroxy group, an alkyl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an aryl group, and a heteroaryl group;

(ii) when B is a benzene ring, —X— and —Y— both represent —NH—, —Z— is —CH₂—, and —W— is —NH—, —A(R²)(R³)(R⁴) is none of a phenyl group, a 4-bromophenyl group, a 4-hydroxyphenyl group, a 4-methoxyphenyl group, a 2-hydroxyphenyl group, a 3,4-dimethoxyphenyl group, or a 3-methoxy-4-hydroxyphenyl group;

(iii) when B is a benzene ring, —X— is —NH—, —Z— is —CR⁶R⁷—, and —W— is —NH—, neither of R⁶ and R⁷ is a methyl group;

(iv) when B is a benzene ring, —X— is —NH—, —Z— is —CO—, and —W— is —NR¹—, R¹ is not a p-tolyl group;

(v) when B is a benzene ring, —X— is —NH—, —Y— is —S—, —Z— is —CR⁶R⁷—, and —W— is —NH—, neither of R⁶ and R⁷ is a methyl group; and

(vi) when B is a benzene ring, —X— is —NH—, —Y— is —S—, and —Z— is —CH₂—, —W— is not —O—.

[15] A compound of Formula (1) or a salt thereof, used for treating a corneal epithelium disorder:

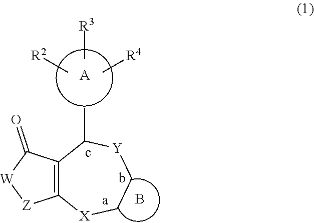

wherein A is an aromatic ring, a heterocyclic ring, or an aliphatic ring; R², R³, and R⁴, which may be identical or different, each independently are a hydrogen atom, a halogen atom, a hydroxy group, an alkyl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an alkenyl group which may have a substituent, an alkynyl group which may have a substituent, an aryl group which may have a substituent, heteroaryl group which may have a substituent, a benzyloxy group which may have a substituent, an aryloxy group which may have a substituent, a heteroaryloxy group which may have a substituent, an arylamino group which may have a substituent, an arylvinyl group which may have a substituent, or an arylethynyl group which may have a substituent; B is an aromatic ring which may have a substituent, a heterocyclic ring which may have a substituent, or an aliphatic ring which may have a substituent; —X—, —Y—, and —Z—, which may be identical or different, each independently are —O—, —NH—, —NR⁵—, —S—, —SO—, —SO₂—, —CH₂—, —CR⁶R⁷—, or —CO— (wherein R⁵ is a lower alkyl group which may have a substituent, an acyl group which may have a substituent, an alkoxycarbonyl group which may have a substituent, a carbamoyl group which may have a substituent, or a sulfonyl group which may have a substituent; and R⁶ and R⁷, which may be identical or different, each independently are a hydrogen atom, a halogen atom, a hydroxy group, an alkyl group which may have a substituent, an aryl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, or a trifluoromethyl group); —W— is —NR¹—, —O—, or —CR⁸R⁹— (wherein R¹ is a hydrogen atom, a lower alkyl group which may have a substituent, or an aryl group which may have a substituent; and R⁸ and R⁹, which may be identical or different, each independently are a hydrogen atom, a halogen atom, a hydroxy group, an alkyl group, an aryl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, or a trifluoromethyl group; and a, b, and c each represent the position of a carbon atom; provided that:

(i) the substituent is selected from the group consisting of a halogen atom, a hydroxy group, an alkyl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an aryl group, and a heteroaryl group;

(ii) when B is a benzene ring, —X— and —Y— both represent —NH—, —Z— is —CH₂—, and —W— is —NH—, —A(R²)(R³)(R⁴) is none of a phenyl group, a 4-bromophenyl group, a 4-hydroxyphenyl group, a 4-methoxyphenyl group, a 2-hydroxyphenyl group, a 3,4-dimethoxyphenyl group, or a 3-methoxy-4-hydroxyphenyl group;

(iii) when B is a benzene ring, —X— is —NH—, —Z— is —CR⁶R⁷—, and —W— is —NH—, neither of R⁶ and R⁷ is a methyl group;

(iv) when B is a benzene ring, —X— is —NH—, —Z— is —CO—, and —W— is —NR¹—, R¹ is not a p-tolyl group;

(v) when B is a benzene ring, —X— is —NH—, —Y— is —S—, —Z— is —CR⁶R⁷—, and —W— is —NH—, neither of R⁶ and R⁷ is a methyl group; and

(vi) when B is a benzene ring, —X— is —NH—, —Y— is —S—, and —Z— is —CH₂—, —W— is not —O—.

[16] The compound according to [15] or a salt thereof, wherein in Formula (1), A is a benzene ring; B is a cyclohexane ring; W is —NR¹—; X is —NH—; Y is —NR⁵—; and Z is —CH₂—.

[17] The compound according to [16] or a salt thereof, wherein R¹ is H, and R⁵ is an acyl group which may have a substituent

[18] The compound according to any one of [15] to [17] or a salt thereof, wherein the compound of Formula (1) is a compound of the following Formula (1a);

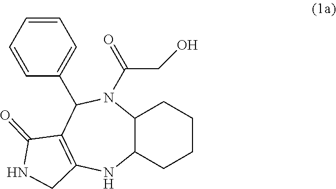 [SMILES: O=C1NCC2=C1C(c1ccccc1)N(C(=O)CO)C1CCCCC1N2]

[19] The compound according to any one of [14] to [18] or a salt thereof, wherein the compound is an eye drop.

[20] The compound according to any one of [14] to [19] or a salt thereof, wherein the corneal epithelium disorder is dry eye.

[21] A compound of Formula (1) or a salt thereof, for suppressing corneal epithelial cell apoptosis:

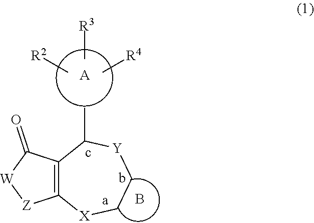

wherein A is an aromatic ring, a heterocyclic ring, or an aliphatic ring; R², R³, and R⁴, which may be identical or different, each independently are a hydrogen atom, a halogen atom, a hydroxy group, an alkyl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an alkenyl group which may have a substituent, an alkynyl group which may have a substituent, an aryl group which may have a substituent, a heteroaryl group which may have a substituent, a benzyloxy group which may have a substituent, an aryloxy group which may have a substituent, a heteroaryloxy group which may have a substituent, an arylamino group which may have a substituent, an arylvinyl group which may have a substituent, or an arylethynyl group which may have a substituent; B is an aromatic ring which may have a substituent, a heterocyclic ring which may have a substituent, or an aliphatic ring which may have a substituent; —X—, —Y—, and —Z—, which may be identical or different, each independently are —O—, —NH—, —NR⁵—, —S—, —SO—, —SO₂—, —CH₂—, —CR⁶R⁷—, or —CO— (wherein R⁵ is a lower alkyl group which may have a substituent, an acyl group which may have a substituent, an alkoxycarbonyl group which may have a substituent, a carbamoyl group which may have a substituent, or a sulfonyl group which may have a substituent; and R⁶ and R⁷, which may be identical or different, each independently are a hydrogen atom, a halogen atom, a hydroxy group, an alkyl group which may have a substituent, an aryl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, or a trifluoromethyl group); —W— is —NR¹—, —O—, or —CR⁸R⁹— (wherein R¹ is a hydrogen atom, a lower alkyl group which may have a substituent, or an aryl group which may have a substituent; and R⁸ and R⁹, which may be identical or different, each independently are a hydrogen atom, a halogen atom, a hydroxy group, an alkyl group, an aryl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, or a trifluoromethyl group); and a, b, and c each represent the position of a carbon atom; provided that:

(i) the substituent is selected from the group consisting of a halogen atom, a hydroxy group, an alkyl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an aryl group, and a heteroaryl group;

(ii) when B is a benzene ring, —X— and —Y— both represent —NH—, —Z— is —CH₂—, and —W— is —NH—, —A(R²)(R³)(R⁴) is none of a phenyl group, a 4-bromophenyl group, a 4-hydroxyphenyl group, a 4-methoxyphenyl group, a 2-hydroxyphenyl group, a 3,4-dimethoxyphenyl group, or a 3-methoxy-4-hydroxyphenyl group;

(iii) when B is a benzene ring, —X— is —NH—, —Z— is —CR⁶R⁷—, and —W— is —NH—, neither of R⁶ and R⁷ is a methyl group;

(iv) when B is a benzene ring, —X— is —NH—, —Z— is —CO—, and —W— is —NR¹, R¹ is not a p-tolyl group;

(v) when B is a benzene ring, —X— is —NH—, —Y— is —S—, —Z— is —CR⁶R⁷—, and —W— is —NH—, neither of R⁶ and R⁷ is a methyl group; and

(vi) when B is a benzene ring, —X— is —NH—, —Y— is —S—, and —Z— is —CH₂—, —W— is not —O—.

[22] A method for treating a corneal epithelium disorder, the method comprising administering an effective amount of a compound of Formula (1) or a salt thereof:

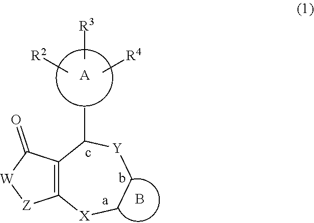

wherein A is an aromatic ring, a heterocyclic ring, or an aliphatic ring; R², R³, and R⁴, which may be identical or different, each independently are a hydrogen atom, a halogen atom, a hydroxy group, an alkyl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an alkenyl group which may have a substituent, an alkynyl group which may have a substituent, an aryl group which may have a substituent, a heteroaryl group which may have a substituent, a benzyloxy group which may have a substituent, an aryloxy group which may have a substituent, a heteroaryloxy group which may have a substituent, an arylamino group which may have a substituent, an arylvinyl group which may have a substituent, or an arylethynyl group which may have a substituent; B is an aromatic ring which may have a substituent, a heterocyclic ring which may have a substituent, or an aliphatic ring which may have a substituent; —X—, —Y—, and —Z—, which may be identical or different, each independently are —O—, —NH—, —NR⁵—, —S—, —SO—, —SO₂—, —CH₂—, —CR⁶R⁷—, or —CO— (wherein R⁵ is a lower alkyl group which may have a substituent, an acyl group which may have a substituent, an alkoxycarbonyl group which may have a substituent, a carbamoyl group which may have a substituent, or a sulfonyl group which may have a substituent; and R⁶ and R⁷, which may be identical or different from each other, each independently are a hydrogen atom, a halogen atom, a hydroxy group, an alkyl group which may have a substituent, an aryl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, or a trifluoromethyl group); —W— is —NR¹—, —O—, or —CR⁸R⁹— (wherein R¹ is a hydrogen atom, a lower alkyl group which may have a substituent, or an aryl group which may have a substituent; and R⁸ and R⁹, which may be identical or different, each independently are a hydrogen atom, a halogen atom, a hydroxy group, an alkyl group, an aryl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, or a trifluoromethyl group; and a, b, and c each represent the position of a carbon atom; provided that:

(i) the substituent is selected from the group consisting of a halogen atom, a hydroxy group, an alkyl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an aryl group, and a heteroaryl group;

(ii) when B is a benzene ring, —X— and —Y— both represent —NH—, —Z— is —CH₂—, and —W— is —NH—, —A(R²)(R³)(R⁴) is none of a phenyl group, a 4-bromophenyl group, a 4-hydroxyphenyl group, a 4-methoxyphenyl group, a 2-hydroxyphenyl group, a 3,4-dimethoxyphenyl group, or a 3-methoxy-4-hydroxyphenyl group;

(iii) when B is a benzene ring, —X— is —NH—, —Z— is —CR⁶R⁷—, and —W— is —NH—, neither of R⁶ and R⁷ is a methyl group;

(iv) when B is a benzene ring, —X— is —NH—, —Z— is —CO—, and —W— is —NR¹—, R¹ is not a p-tolyl group;

(v) when B is a benzene ring, —X— is —NH—, —Y— is —S—, —Z— is —CR⁶R⁷—, and —W— is —NH—, neither of R⁶ and R⁷ is a methyl group; and

(vi) when B is a benzene ring, —X— is —NH—, —Y— is —S—, and —Z— is —CH₂—, —W— is not —O—.

[23] The method according to [22], wherein in Formula (1), A is a benzene ring; B is a cyclohexane ring; W is —NR¹—; X is —NH—; Y is —NR⁵—; and Z is —CH₂—.

[24] The method according to [23], wherein R¹ is H, and R⁵ is an acyl group which may have a substituent.

[25] The method according to any one of [22] to [24], wherein the compound of Formula (1) is a compound of the following Formula (1a):

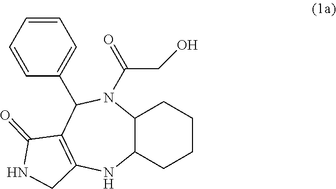 [SMILES: O=C1NCC2=C1C(c1ccccc1)N(C(=O)CO)C1CCCCC1N2]

[26] The method according to any one of [22] to [25], wherein the route of administration is instillation.

[27] The method according to any one of [22] to [26], wherein the corneal epithelium disorder is dry eye.

[28] A method for suppressing corneal epithelial cell apoptosis, the method comprising administering an effective amount of a compound of Formula (1) or a salt thereof:

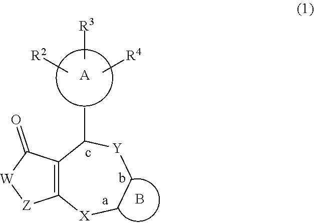

wherein A is an aromatic ring, a heterocyclic ring, or an aliphatic ring; R², R³, and R⁴, which may be identical or different, each independently are a hydrogen atom, a halogen atom, a hydroxy group, an alkyl group, a mercapto group, an alkoxy group, an alkylthiogroup, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an alkenyl group which may have a substituent, an alkynyl group which may have a substituent, an aryl group which may have a substituent, a heteroaryl group which may have a substituent, a benzyloxy group which may have a substituent, an aryloxy group which may have a substituent, a heteroaryloxy group which may have a substituent, an arylamino group which may have a substituent, an arylvinyl group which may have a substituent, or an arylethynyl group which may have a substituent; B is an aromatic ring which may have a substituent, a heterocyclic ring which may have a substituent, or an aliphatic ring which may have a substituent; —X—, —Y—, and —Z—, which may be identical or different, each independently are —O—, —NH—, —NR⁵—, —S—, —SO—, —SO₂—, —CH₂—, —CR⁶H⁷— or —CO— (wherein R⁵ is a lower alkyl group which may have a substituent, an acyl group which may have a substituent, an alkoxycarbonyl group which may have a substituent, a carbamoyl group which may have a substituent, or a sulfonyl group which may have a substituent; and R⁶ and R⁷, which may be identical or different, each independently are a hydrogen atom, a halogen atom, a hydroxy group, an alkyl group which may have a substituent, an aryl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, or a trifluoromethyl group); —W— is —NR¹—, —O—, or —CR⁸R⁹— (wherein R¹ is a hydrogen atom, a lower alkyl group which may have a substituent, or an aryl group which may have a substituent; R⁸ and R⁹, which may be identical or different, each independently are a hydrogen atom, a halogen atom, a hydroxy group, an alkyl group, an aryl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, or a trifluoromethyl group); and a, b, and c each represent the position of a carbon atom; provided that:

(i) the substituent is selected from the group consisting of a halogen atom, a hydroxy group, an alkyl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an aryl group, and a heteroaryl group;

(ii) when B is a benzene ring, —X— and —Y— both represent —NH—, —Z— is —CH₂—, and —W— is —NH—, —A(R²)(R³)(R⁴) is none of a phenyl group, a 4-bromophenyl group, a 4-hydroxyphenyl group, a 4-methoxyphenyl group, a 2-hydroxyphenyl group, a 3,4-dimethoxyphenyl group, or a 3-methoxy-4-hydroxyphenyl group;

(iii) when B is a benzene ring, —X— is —NH—, —Z— is —CR⁶R⁷—, and —W— is —NH—, neither of R⁶ and R⁷ is a methyl group;

(iv) when B is a benzene ring, —X— is —NH—, —Z— is —CO—, and —W— is —NR¹—, R¹ is not a p-tolyl group;

(v) when B is a benzene ring, —X— is —NH—, —Y— is —S—, —Z— is —CR⁶R⁷—, and —W— is —NH—, neither of R⁶ and R⁷ is a methyl group; and

(vi) when B is a benzene ring, —X— is —NH—, —Y— is —S—, and —Z— is —CH₂—, —W— is not —O—.

### Advantageous Effects of Invention

The agent for the treatment of a corneal epithelium disorder of the present invention suppresses apoptosis of corneal epithelial cells and also has an effect of increasing lacrimal fluid and an effect of increasing mucin. The agent for the treatment of a corneal epithelium disorder improves symptoms of the dry eye and also enables radical cure of the dry eye.

### BRIEF DESCRIPTION OF DRAWINGS

FIG. 1 illustrates a decrease in the survival rate of corneal epithelial cells caused by hyperosmotic stress (addition of NaCl).

FIG. 2 illustrates the effects of Compound (1) of the present invention on the decrease in the survival rate of corneal epithelial cells caused by hyperosmotic stress (addition of NaCl).

FIG. 3 illustrates the effects of Compound (1) of the present invention on the decrease in the survival rate of corneal epithelial cells caused by hyperosmotic stress (addition of glucose).

FIG. 4 illustrates the effects of Compound (1) of the present invention on the decrease in the survival rate of corneal epithelial cells caused by ethanol.

FIG. 5 illustrates the effects of Compound (1) of the present invention on the hyperosmotic stress (addition of NaCl)-dependent apoptosis of corneal epithelial cells.

FIG. 6 illustrates the effects of Compound (1) of the present invention on the amount of lacrimal fluid in a dry eye model rat.

FIG. 7 illustrates the effects of Compound (1) of the present invention on the injury of corneal surface in a dry eye model rat.

FIG. 8 illustrates the effects of Compound (1) of the present invention on the expression of a mucin-producing gene in corneal epithelial cells.

### DESCRIPTION OF EMBODIMENTS

An active ingredient of the agent for the treatment of a corneal epithelium disorder of the present invention is a compound of Formula (1) or a salt thereof. This compound is a compound described in WO 2002/044180 A, and is known to be useful as an agent for the treatment of diabetes mellitus. However, nothing is known about the action of the compound on corneal epithelium disorders.

In Formula (1), A is an aromatic ring, a heterocyclic ring, or an aliphatic ring; however, an aromatic ring is preferred, while a benzene ring is more preferred.

R², R³, and R⁴, which may be identical or different, each independently are a hydrogen atom, a halogen atom, a hydroxy group, an alkyl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an alkenyl group which may have a substituent, an alkynyl group which may have a substituent, an aryl group which may have a substituent, a heteroaryl group which may have a substituent, a benzyloxy group which may have a substituent, an aryloxy group which may have a substituent, a heteroaryloxy group which may have a substituent, an arylamino group which may have a substituent, an arylvinyl group which may have a substituent, or an arylethynyl group which may have a substituent. However, a hydrogen atom, a halogen atom, a hydroxy group, an alkyl group, an alkoxy group, an alkylthio group, a carboxy group, or an alkoxycarbonyl group are preferred. Furthermore, a case in which R², R³, and R⁴ all are a hydrogen atom is particularly preferred.

B is an aromatic ring which may have a substituent, heterocyclic ring which may have a substituent, or an aliphatic ring which may have a substituent. However, an aliphatic ring which may have a substituent is preferred, a cycloalkane ring having 5 or 6 carbon atoms is more preferred, and a cyclohexane ring is even more preferred.

X, Y, and Z each independently are —O—, —NH—, NR⁵—, —S—, —SO—, —SO₂—, —CH₂—, —CR⁶R⁷—, or —CO—. However, X is preferably —NH—, Y is preferably —NR⁵—, and Z is preferably —CH₂—. Here, is a lower alkyl group which may have a substituent, an acyl group which may have a substituent, an alkoxycarbonyl group which may have a substituent, a carbamoyl group which may have a substituent, or a sulfonyl group which may have a substituent. However, a lower alkyl group, or an acyl group which may have s substituent is preferred; an alkanoyl group which may have a substituent is more preferred; a C₂-C₄ alkanoyl group which may have a substituent is even more preferred; and an acetyl group which may have a substituent is still more preferred.

(i) The substituent described above is selected from the group consisting of a halogen atom, a hydroxy group, an alkyl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an aryl group, and a heteroaryl group. Among these, a halogen atom, a hydroxy group, a mercapto group, an alkoxy group, or an alkylthio group is preferred; a hydroxy group or an alkoxy group is more preferred; and a hydroxy group is even more preferred.

W is —NR¹—, —O—, or —CR⁸R⁹—; however, among these, —NR¹— is preferred. Here, R¹ is preferably a hydrogen atom or a lower alkyl group, and more preferably a hydrogen atom.

A compound in which in Formula (1), A is a benzene ring; B is a cyclohexane ring; W is —N¹—; X is —NH—; Y is —NR⁵—; and Z is —CH₂—, is more preferred. Also, here, a compound in which R¹ is H; and R⁵ is an acyl group which may have a substituent, is even more preferred.

Among compounds of Formula (1), a compound of the following Formula (1a) is more preferred:

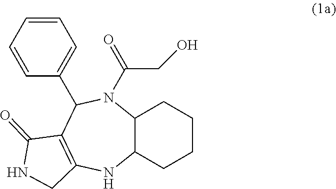 [SMILES: O=C1NCC2=C1C(c1ccccc1)N(C(=O)CO)C1CCCCC1N2]

Furthermore, a compound of the following Formula (1aa) [(1R,8R,10R)-9-(2-hydroxyacetyl)-8-phenyl-2,5,9-triazatricyclo[8.4.0.0^{3,7}]tetradeca-3(7)-en-6-one] is particularly preferred.

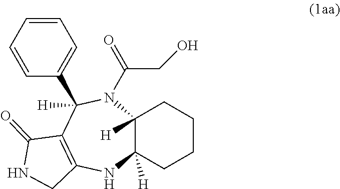

Examples of a salt of the compound of Formula (1) (hereinafter, maybe referred to as Compound (1) of the present invention) include basic salts such as an ammonium salt, an alkali metal salt (for example, sodium salt, potassium salt), and an alkaline earth metal salt (for example, calcium salt, magnesium salt); and acid addition salts such as hydrochloride, sulfate, nitrate, acetate, lactate, and citrate.

Furthermore, Compound (1) of the present invention has an asymmetric carbon atom and thus has optical isomers. Therefore, optically active substances are also included therein. Moreover, Compound (1) of the present invention or a salt thereof may also be in the form of a hydrate or a solvate.

Compound (1) of the present invention or a salt thereof can be produced according to the method described in WO 2002/044180 A.

As illustrated in the Examples described below, Compound (1) of the present invention or a salt thereof is useful as an agent for the treatment of corneal epithelium disorders because the compound suppresses a decrease in the survival rate of corneal epithelial cells caused by hyperosmotic stress, suppresses the hyperosmotic stress-dependent apoptosis of corneal epithelial cells, and suppresses corneal injury. Corneal epithelium disorders include corneal epithelium disorders involved in infectious diseases caused by inappropriate use of contact lenses, and chemistry of eye drop antiseptic agents, autoimmune diseases such as Sjögren's syndrome, diabetes mellitus, and dry eye. Since Compound (1) or a salt thereof has an effect of increasing the amount of lacrimal fluid and the amount of mucin, Compound (1) or a salt thereof is useful particularly as an agent for the treatment of dry eye.

Examples of the dosage form for the agent for the treatment of corneal epithelium disorders of the present invention include an eye drop, an injectable preparation, an oral preparation (tablets, a granular preparation, a powder, and capsules), an ointment, and a cream. Among these, an eye drop is particularly preferred. In order to produce the agent into the form of these pharmaceutical compositions, the agent can be formulated together with a pharmaceutically acceptable carrier. Examples of such a carrier include lactose, glucose, D-mannitol, starch, crystalline cellulose, calcium carbonate, kaolin, starch, gelatin, hydroxypropyl cellulose, hydroxypropylmethyl cellulose, poly-vinylpyrrolidone, ethanol, carboxymethyl cellulose, carboxymethyl cellulose calcium salt, magnesium stearate, talc, acetyl cellulose, white sugar, titanium oxide, benzoic acid, paraoxybenzoic acid ester, sodium dehydroacetate, gum arabic, tragacanth, methyl cellulose, egg yolk, surfactants, white sugar, simple syrup, citric acid, distilled water, ethanol, glycerin, propylene glycol, macrogol, sodium hydrogen phosphate, sodium dihydrogen phosphate, sodium phosphate, glucose, sodium chloride, phenol, thimerosal, para-oxybenzoic acid ester, and sodium hydrogen sulfite. These carriers are used in appropriate mixtures with Compound (1) of the present invention in accordance with the dosage form.

When the pharmaceutical composition of the present invention is produced into an eye drop, for example, sodium hyaluronate, diquafosol sodium, and rebamipide can also be incorporated in addition to Compound (1) of the present invention or a salt thereof.

Furthermore, the content of the active ingredient of the present invention in the pharmaceutical composition preparation of the present invention greatly depends on the form of the preparation and is not particularly limited. However, the content is usually 0.01% to 100% by weight, and preferably 1% to 100% by weight, with respect to the total amount of the composition.

The dosage of the therapeutic agent for corneal epithelium disorders of the present invention may depend on the symptoms and age of the patient in need of administration, and the method for administration; however, the dosage is preferably from 0.01 to 100 mg/kg/day.

### EXAMPLES

Next, the present invention will be described in more detail by way of Examples.

### Example 1

### (Suppressive Effect on Cell Injury Under Hyperosmotic Conditions)

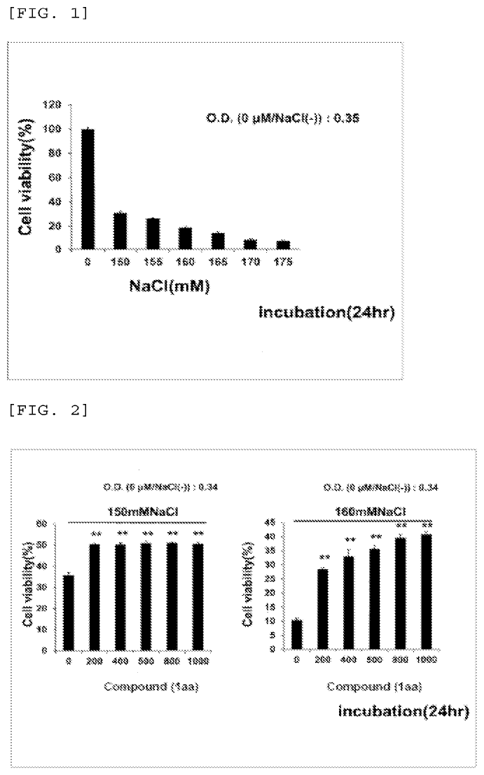

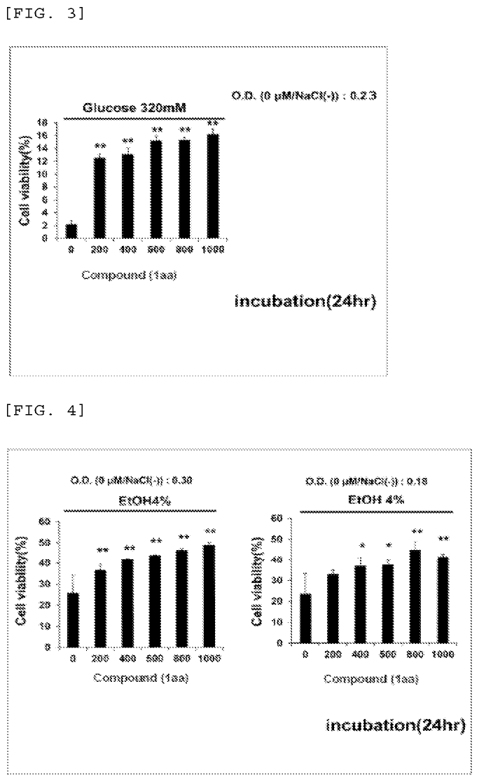

HCE cells (human corneal epithelial cells) were cultured for 24 hours in a hyperosmotic medium containing a compound of Formula (1aa) (hereinafter, Compound (1aa)) (as a hydrate). The medium was adjusted to fulfill the hyperosmotic conditions using 150 mM NaCl and 160 mM NaCl, or 320 mM glucose. The number of live cells was measured by the MTT method, and relative values of light absorbance with respect to the light absorbance of the control (isotonic pressure conditions) were calculated. The results are presented in FIG. 1 to FIG. 3. The value is expressed as average value±S.D. (n=3), **P<0.01.

As can be seen from FIG. 1 to FIG. 3, Compound (1aa) noticeably suppressed the decrease in the survival rate of corneal epithelial cells caused by the hyperosmotic conditions.

### Example 2

### (Suppressive Effect on Cell Injury Caused by Ethanol)

HCE cells (human corneal epithelial cells) were cultured in a medium containing Compound (1aa) and 4 v/v % ethanol. The number of live cells was measured by the MTT method, and relative values of light absorbance with respect to the light absorbance of the control (absence of ethanol) were calculated. The results are presented in FIG. 4. The value is expressed as average value±S.D. (n=3), *P<0.05; and **P<0.01.

As can be seen from FIG. 4, Compound (1aa) noticeably suppressed the decrease in the survival rate of corneal epithelial cells caused by ethanol.

### Example 3

### (Suppressive Effect on Apoptosis Under Hyperosmotic Conditions)

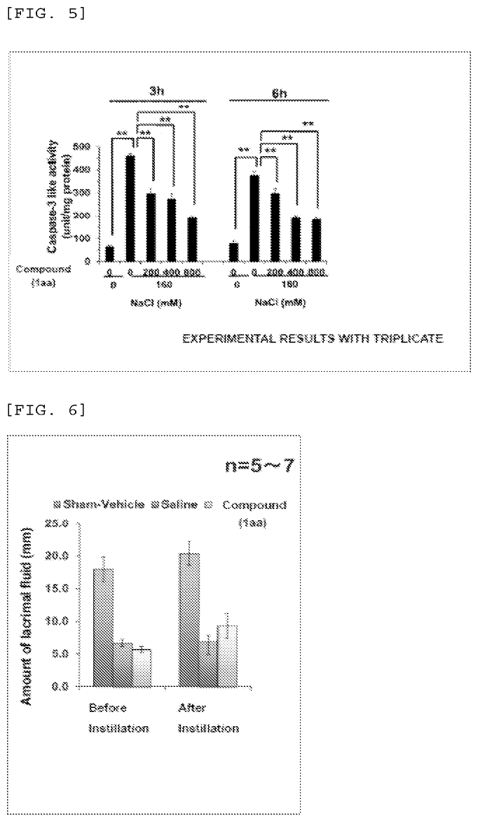

HCE cells were cultured in a hyperosmotic medium containing Compound (1aa) and 160 mM NaCl. After culturing for 3 hours and 6 hours, caspase-3-like activity, which is a marker for apoptosis, was measured using a fluorescent peptide substrate, and the results are presented in FIG. 5. The value is expressed as average value±S.D. (triplicate), **P<0.01, and the term n.s. means “not significant”.

As can be seen from FIG. 5, Compound (1aa) noticeably suppressed hyperosmotic stress-dependent apoptosis of corneal epithelial cells.

### Example 4

### (Therapeutic Effect on Corneal Surface Injury in Rat)

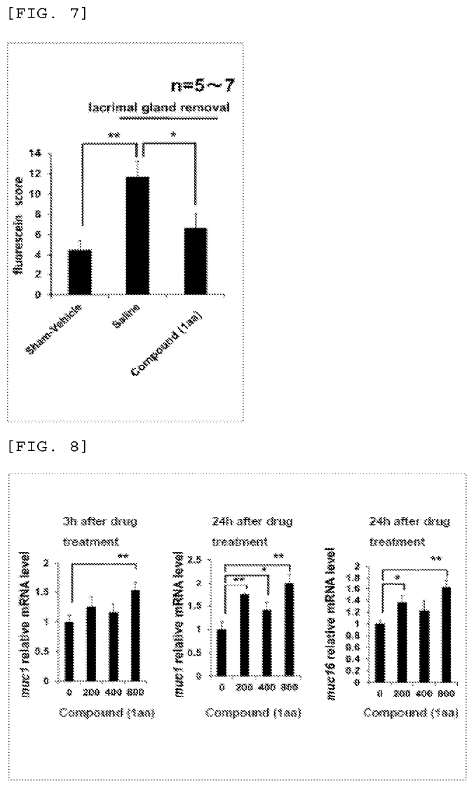

The lacrimal glands of a rat were removed, and a dry eye model was produced. After 1 to 5 weeks from the removal of lacrimal glands, 5 μL of an aqueous solution containing Compound (1aa) (0.05 w/v %, 1.5 mM) was administered to the rat three times a day. The amount of lacrimal fluid was measured by a cotton thread test, and the results are presented in FIG. 6. An image of cornea stained with fluorescein is presented in FIG. 7. The scores of fluorescein were calculated, and the results are presented in FIG. 7. The value is expressed as average value±S.E.M., *P<0.05; and **P<0.01.

As can be seen from FIG. 6 and FIG. 7, Compound (1aa) suppressed injury of the corneal surface by increasing the amount of lacrimal fluid in the dry eye model rat.

### Example 5

### (Change in Amount of Expression of Messenger RNA (Effect on Mucin Expression))

HCE cells were cultured in a medium containing Compound (1aa). The amounts of expression of muc1 and muc16 messenger RNAs after culturing for 3 hours and 24 hours were measured by a real time RT-PCR method. Relative values of the amount of expression with respect to the amount of expression of actin messenger RNA were calculated, and the results are presented in FIG. 8. The value is expressed as average value±S.D. (triplicate), *P<0.05; and **P<0.01.

As can be seen from FIG. 8, it became clear that Compound (1aa) increases the amount of expression of mucin in corneal epithelial cells.

1-21. (canceled)

22. A method for treating a corneal epithelium disorder, the method comprising:
administering an effective amount of a compound of Formula (1) or a salt thereof: 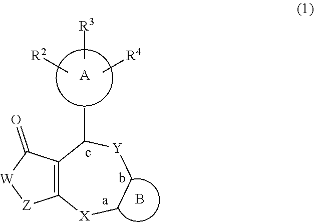
wherein A is an aromatic ring, a heterocyclic ring, or an aliphatic ring; R², R³, and R⁴, which are identical or different, each independently are a hydrogen atom, a halogen atom, a hydroxy group, an alkyl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an alkenyl group which optionally has a substituent, an alkynyl group which optionally has a substituent, an aryl group which optionally has a substituent, a heteroaryl group which optionally has a substituent, a benzyloxy group which optionally has a substituent, an aryloxy group which optionally has a substituent, a heteroaryloxy group which optionally has a substituent, an arylamino group which optionally has a substituent, an arylvinyl group which optionally has a substituent, or an arylethynyl group which optionally has a substituent; B is an aromatic ring which optionally has a substituent, a heterocyclic ring which optionally has a substituent, or an aliphatic ring which optionally has a substituent; —X—, —Y—, and —Z—, which are identical or different, each independently are —O—, —NH—, —NR⁵—, —S—, —SO—, —SO₂—, —CH₂—, —CR⁶R⁷—, or —CO— (wherein R⁵ is a lower alkyl group which optionally has a substituent, an acyl group which optionally has a substituent, an alkoxycarbonyl group which optionally has a substituent, a carbamoyl group which optionally has a substituent, or a sulfonyl group which optionally has a substituent; and R⁶ and R⁷, which are identical or different, each independently are a hydrogen atom, a halogen atom, a hydroxy group, an alkyl group which optionally has a substituent, an aryl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, or a trifluoromethyl group); —W— is —NR¹—, —O—, or —CR⁸R⁹— (wherein R¹ is a hydrogen atom, a lower alkyl group which optionally has a substituent, or an aryl group which optionally has a substituent; and R⁸ and R⁹, which are identical or different, each independently are a hydrogen atom, a halogen atom, a hydroxy group, an alkyl group, an aryl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, or a trifluoromethyl group; and a, b, and c each represent the position of a carbon atom; provided that:
(i) the substituent is selected from the group consisting of a halogen atom, a hydroxy group, an alkyl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an aryl group, and a heteroaryl group;
(ii) when B is a benzene ring, —X— and —Y— both represent —NH—, —Z— is —CH₂—, and —W— is —NH—, —A(R²)(R³)(R⁴) is none of a phenyl group, a 4-bromophenyl group, a 4-hydroxyphenyl group, a 4-methoxyphenyl group, a 2-hydroxyphenyl group, a 3,4-dimethoxyphenyl group, or a 3-methoxy-4-hydroxyphenyl group;
(iii) when B is a benzene ring, —X— is —NH—, —Z— is —CR⁶R⁷—, and —W— is —NH—, neither of R⁶ and R⁷ is a methyl group;
(iv) when B is a benzene ring, —X— is —NH—, —Z— is —CO—, and —W— is —NR¹—, R¹ is not a p-tolyl group;
(v) when B is a benzene ring, —X— is —NH—, —Y— is —S—, —Z— is —CR⁶R⁷—, and —W— is —NH—, neither of R⁶ and R⁷ is a methyl group; and
(vi) when B is a benzene ring, —X— is —NH—, —Y— is —S—, and —Z— is —CH₂—, —W— is not —O—.

23. The method according to claim 22, wherein in Formula (1), A is a benzene ring; B is a cyclohexane ring; W is —NR¹—; X is —NH—; Y is —NR⁵—; and Z is —CH₂—.

24. The method according to claim 23, wherein R¹ is H, and R⁵ is an acyl group which optionally has a substituent.

25. The method according to claim 22, wherein the compound of Formula (1) is a compound of Formula (1a):

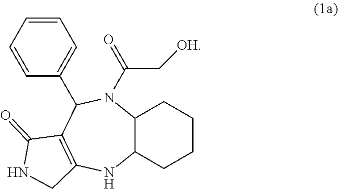 [SMILES: O=C1NCC2=C1C(c1ccccc1)N(C(=O)CO)C1CCCCC1N2]

26. The method according to claim 22, wherein a route of the administration is instillation.

27. The method according to claim 22, wherein the corneal epithelium disorder is dry eye.

28. A method for suppressing corneal epithelial cell apoptosis, the method comprising:
administering an effective amount of a compound of Formula (1) or a salt thereof: 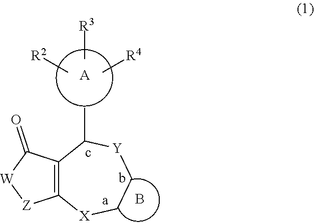
wherein A is an aromatic ring, a heterocyclic ring, or an aliphatic ring; R², R³, and R⁴, which are identical or different, each independently are a hydrogen atom, a halogen atom, a hydroxy group, an alkyl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an alkenyl group which optionally has a substituent, an alkynyl group which optionally has a substituent, an aryl group which optionally has a substituent, a heteroaryl group which optionally has a substituent, a benzyloxy group which optionally has a substituent, an aryloxy group which optionally has a substituent, a heteroaryloxy group which optionally has a substituent, an arylamino group which optionally has a substituent, an arylvinyl group which optionally has a substituent, or an arylethynyl group which optionally has a substituent; B is an aromatic ring which optionally has a substituent, a heterocyclic ring which optionally has a substituent, or an aliphatic ring which optionally has a substituent; —X—, —Y—, and —Z—, which are identical or different, each independently are —O—, —NH—, —NR⁵—, —S—, —SO—, —SO₂—, —CH₂—, —CR⁶R⁷—, or —CO— (wherein R⁵ is a lower alkyl group which optionally has a substituent, an acyl group which optionally has a substituent, an alkoxycarbonyl group which optionally has a substituent, a carbamoyl group which optionally has a substituent, or a sulfonyl group which optionally has a substituent; and R⁶ and R⁷, which are identical or different, each independently are a hydrogen atom, a halogen atom, a hydroxy group, an alkyl group which optionally has a substituent, an aryl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, or a trifluoromethyl group); —W— is —NR¹—, —O—, or —CR⁸R⁹— (wherein R¹ is a hydrogen atom, a lower alkyl group which optionally has a substituent, or an aryl group which optionally has a substituent; R⁸ and R⁹, which are identical or different, each independently are a hydrogen atom, a halogen atom, a hydroxy group, an alkyl group, an aryl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, or a trifluoromethyl group); and a, b, and c each represent the position of a carbon atom; provided that:
(i) the substituent is selected from the group consisting of a halogen atom, a hydroxy group, an alkyl group, a mercapto group, an alkoxy group, an alkylthio group, an alkylsulfonyl group, an acyl group, an acyloxy group, an amino group, an alkylamino group, a carboxy group, an alkoxycarbonyl group, a carbamoyl group, a nitro group, a cyano group, a trifluoromethyl group, an aryl group, and a heteroaryl group;
(ii) when B is a benzene ring, —X— and —Y— both represent —NH—, —Z— is —CH₂—, and —W— is —NH—, —A(R²)(R³)(R⁴) is none of a phenyl group, a 4-bromophenyl group, a 4-hydroxyphenyl group, a 4-methoxyphenyl group, a 2-hydroxyphenyl group, a 3,4-dimethoxyphenyl group, or a 3-methoxy-4-hydroxyphenyl group;
(iii) when B is a benzene ring, —X— is —NH—, —Z— is —CR⁶R⁷—, and —W— is —NH—, neither of R⁶ and R⁷ is a methyl group;
(iv) when B is a benzene ring, —X— is —NH—, —Z— is —CO—, and —W— is —NR¹—, R¹ is not a p-tolyl group;
(v) when B is a benzene ring, —X— is —NH—, —Y— is —S—, —Z— is —CR⁶R⁷—, and —W— is —NH—, neither of R⁶ and R⁷ is a methyl group; and
(vi) when B is a benzene ring, —X— is —NH—, —Y— is —S—, and —Z— is —CH₂—, —W— is not —O—.
